(19) United States
(12) Patent Application Publication
(10) Pub. No.: US 2012/0065438 A1
(43) Pub. Date: Mar. 15, 2012

(54) Process for Syntheis of 10,10-Dibromo-9,9-dianthracene

(76) Inventors: Lifei Cai, Beijing (CN); Lei Dai, Beijing (CN); Hongyu Zhao, Beijing (CN)

(21) Appl. No.: 13/321,774

(22) PCT Filed: May 5, 2010
(86) PCT No.: PCT/CN10/72443

§ 371 Date: Nov. 21, 2011

(30) Foreign Application Priority Data
May 22, 2009 (CN) 200910085440.0

(51) Int. Cl. C07C 17/06

(52) U.S. Cl. 570/206

(57) ABSTRACT

This invention, which belongs to the field of organic electroluminesent materials synthetic technology, involves synthetic method of 10,10′-Dibromo-9,9′-bianthryl. The synthetic method is to use 9,9′-bianthryl as raw material, chlorinated hydrocarbon as solvent, bromide as bromine agent and react. This method not only produces no environmental pollution, but also enjoys high yield, therefore, it is suitable for industrial production.

### TECHNICAL FIELD

This invention, which belongs to the field of organic electroluminesent materials synthetic technology, involves synthetic method of 10,10′-Dibromo-9,9′-bianthryl, the intermediate of field-effect transistor materials and organic electroluminesent materials.

### TECHNICAL BACKGROUND

In 1987, Ching W. Tang of Kodak Company in the U.S. successfully worked out sandwich-type double-layer light-emitting devices by using Alq₃ as luminescent layer and aromatic diamines as hole transport layer (Tang C. W., et al. Applied Physics Letters, 1987, 51, 913). In 1990, Burroughes J. H. et al. of Cambridge University developed organic polymer light-emitting diodes (Burroughes J. H. et al. Nartue, 1990, 347, 5395). These major breakthroughs pushed development of organic light emitting technology significantly. OLED products have already been commercialized, its advantages, such as soft colors and high definition, are attracting the attention of an increasing number of people. And its fatal weakness of lifespan and stability can be improved through looking for new light-emitting materials and reforming manufacturing technology. As a result, synthesis and performance of new organic light-emitting materials are the focus of current research. Scientists of various countries have devoted much energy to research and development of this technology, consequently, an increasing number of organic electroluminescent materials have been developed and applied, and among which anthraquinone compounds is a kind of organic light-emitting materials with special luminescence properties and good performance. The band-gap of 9,9′-bianthryl crystal is about 3 eV, only light with wavelength below 410 nm can stimulate it, so it is very stable in the air. In addition, hole mobility of crystal based on bianthryl at room temperature can reach 3 cm²/V·s, therefore, bianthryl field effect transistor has attracted a considerable amount of interests of research recently. Meanwhile, its derivative is a kind of very promising blue light-emitting material (M. H. Ho, Y. S. Wu, S. W. Wen et al., Appl. Phys. Lett., 2006, 89, 252903/1-3), and very important to study and manufacture of blue, white color organic electroluminescent devices.

9,9′-Bianthryl is a kind of very good blue light-emitting material, and its energy level is 3.1 ev (J.-H. Jou, Ch.-P. Wang, et al., Organic Electronics, 2007, 8, 29-36.). Bianthryl derivatives can be synthesized through bianthryl, but the current synthetic method of bianthryl and its dibromide has problems such as low yield and complex after treatment (Yuliang Mai, Guangdong Chemical Industry, 2007, 34, 9; J. Chem. Soc., 1949, 267-269.). It is known by literature that dianthranide was mainly synthesized through anthraquinone, the yield was 40-50% (J. Chem. Soc., 1949, 267-269) which is not suitable for industrialization, through Yuliang Mai improved the synthesis, phosphorus pentoxide was easy to wrap raw materials and many raw materials did not participate in the reaction. According to literatures, 10,10′-Dibromo-9,9′-bianthryl is mainly synthesized by carbon sulfide and carbon tetrachloride (Uwe Mueller, Martin Baumgarten, J. Am. Chem. Soc., 1995, 117 (21), 5840-5850; Yuliang Mai, Guangdong Chemical Industry 2007, 3, 49 J. Chem. Soc., 1949, 267-269.), though appropriate yield is obtained, it is not suitable for industrial production, and meanwhile, its environmental pollution is quite severe. Bianthryl barely dissolve in carbon tetrachloride, and during the reaction, mono-bromine compound will be separated out, its yield is not high as well. Therefore, suitable solvents are needed for dibromide of bianthryl in order to solve the problem of precipitation and cut down reaction time.

### CONTENT OF THIS INVENTION

Aiming at defects in the above field, this invention provides a synthetic method of 10,10′-Dibromo-9,9′-bianthryl, which not only produces no environmental pollution and enjoys high yield, but also reduces reaction time, thus it is suitable for industrialization production.

Synthetic method of 10,10′-Dibromo-9,9′-bianthryl: add 9,9′-bianthryl as raw material, chlorinated hydrocarbon as solvent, bromide as bromine agent and react.

The stated chlorinated hydrocarbon is 1,2-dichloroethane, 1,1-dichloroethane, 1,1,1-trichloroethane, 1,1,2-trichloroethane, tetrachloroethane, 1,1,2,2-tetrachloroethane, pentachloroethane, chloropropane, 1,2-dichloropropane, 1,3-dichloropropane, 1,3-dichloropropane, 1,2,3-trichloropropane, chlorobutane, 2-chlorobutane, 1-chloro-2-methylpropane, 2-chloro-2-methyl-, 1,4-dichlorobutane, chloropentene, tert-pentyl chloride, chloroisopentane, dichloropentane, methylene chloride, 1,5-dichloropentane, chlorobenzene or o-dichlorobenzene.

Drop the stated bromide in the reaction solution at −10° to −80°.

The stated reaction is to react 3-10 hours after addition of bromide at −10° to −80°.

The stated completion of reaction also includes after treatment procedures which consists of filtrating to obtain solids and washing them with solvents.

The stated 9,9′-bianthryl is obtained as follows: add anthraquinone as raw material and zinc as reducing agent in glacial acetic acid solution, then batch addition of hydrochloric acid at 70-120°, maintain the temperature unchanged and react, then 9,9′-bianthryl is achieved.

As stated, the reaction time should be 2-15 hours at 70-120°.

It is optimal to react 2-10 hours at 80-110°.

The stated reaction should take place under protection of nitrogen.

Using bianthryl to produce 10,10′-dibromo-9,9′-bianthryl as stated above is a typical bromination, the reaction cannot be completed in one step because bianthryl was substituted via two bromine simultaneously, if higher yield is needed, then appropriate halogenation solvent needs to be found, which can dissolve both bianthryl and mono complex, and meanwhile, the dibromo-complex cannot dissolve in the solvent in order to better separate it out. For this reason, this invention selects chlorinated hydrocarbon as listed above, and experiments has already showed that they are good halogenation solvents and can separate products out very well, in addition, no mono-bromine compound was obtained during reaction, as a result, yield is improved meanwhile reaction time is reduced. The bianthryl adopted in this invention can also be obtained from other methods, or the method below.

Synthetic method of bianthryl: use acetic acid as solution, anthraquinone as raw material, zinc powder and hydrochloric acid as reducing agent, the synthetic reaction is completed in one step and after treatment is convenient and easy as well.

The compound designed in this patent can be achieved in accordance with the following procedure:

(1) Add acetic acid, zinc powder, anthraquinone in reaction flask, stir and fill it with nitrogen, dropping hydrochloric acid in it while maintaining the temperature between 80°˜110°.

(2) After addition of hydrochloric acid, keep temperature between 80°˜110° and react 2-10 hours, then cool it, separate solids out, filtrate solids and purify them.

(3) Add the bianthryl obtained into chlorinated hydrocarbon, keep temperature at −10°˜80°, then drop bromine in it, after that, react 3˜10 hours. After the reaction is completed, filtrate and purify solids.

The method provided by this invention is simple and reliable with no environmental pollution problem; therefore, it is very suitable for industrial production.

### PRACTICAL IMPLEMENTAION MODES

### Implementation Example 1

Add 600 ml glacial acetic acid, 25 g anthraquinone, and 55 g zinc powder in boiling flask-4-neck, fill it with nitrogen, heat and stir it. Drop 150 ml hydrochloric acid slowly while maintaining the temperature between 80° to 90°. After that, react at 90°, the color of the mixture is growing deep and solids are separated out gradually. After 8-hour reaction, no raw materials are left on the tap, then stop the reaction, filtrate and purify the solids with toluene, after drying them, 17 g products are gained with yield of 80%.

m.p.>300°;

1HNMR (CDCl₃): 7.00˜7.19 (m,8H), 7.42˜7.48 (m,4H), 8.27 (d, J=12, 3 Hz, 4H), 8.67 (s, 2H); ESIMS z/e: 355.1 [M+H]+.

### Implementation Example 2

Add 5 g bianthryl, 80 ml 1,2-dichloroethane in 250 ml boiling flask-4-neck, then drop 5 g bromine, the solution turns red with no significant change of temperature. After addition of bromine, stir it for 4 hours at room temperature, a large amount of solid is separated out, filtrate and wash the solids with 20 ml 1,2-dichloroethane, 6.5 g product is gained with 80% of yield.

m.p.>300°

1HNMR (CDCl₃): 7.05˜7.08 (m, 4H), 7.15˜7.19 (m, 4H), 7.55˜7.59 (m, 4H), 8.68˜8.71 (m, 4H); ESIMS z/e: 510.9 [M+H]+

### Implementation Example 3

Add 5 g bianthryl, 80 ml tetrachloroethane in 250 ml boiling flask-4-neck, then drop 5 g bromine, the solution turns red with no significant change of temperature. After addition of bromine, stir it for 4 hours at room temperature, a large amount of solid is separated out, filtrate and wash the solids with 20 ml tetrachloroethane, 6 g product is gained with 75% of yield.

m.p.>300°

1HNMR (CDCl₃): 7.05˜7.08 (m, 4H), 7.15˜7.19 (m, 4H), 7.55˜7.59 (m, 4H), 8.68˜8.71 (m, 4H); ESIMS z/e: 510.9 [M+H]+

### Implementation Example 4

### O-dichlorobenzene

Add 5 g bianthryl, 80 ml o-dichlorobenzene in 250 ml boiling flask-4-neck, then drop 5 g bromine, the solution turns red with no significant change of temperature. After addition of bromine, stir it for 4 hours at room temperature, a large amount of solid is separated out, filtrate and wash the solids with 20 ml o-dichlorobenzene, 6.2 g product is gained with 78% of yield.

m.p.>300°

1HNMR (CDCl₃): 7.05˜7.08 (m, 4H), 7.15˜7.19 (m, 4H), 7.55˜7.59 (m, 4H), 8.68˜8.71 (m, 4H); ESIMS z/e: 510.9 [M+H]+

1. A method of synthesizing 10,10′-Dibromo-9,9′-bianthryl comprising: adding 9,9′-bianthryl as raw material, chlorinated hydrocarbon as solvent, bromide as bromine agent and reacting, wherein the stated chlorinated hydrocarbon is selected from 1,2-dichloroethane, 1,1-dichloroethane, 1,1,1-trichloroethane, 1,1,2-trichloroethane, tetrachloroethane, 1,1,2,2-tetrachloroethane, pentachloroethane, chloropropane, 1,2-dichloropropane, 1,3-dichloropropane, 1,3-dichloropropane, 1,2,3-trichloropropane, chlorobutane, 2-chlorobutane, 1-chloro-2-methylpropane, 2-chloro-2-methyl-, 1,4-dichlorobutane, chloropentene, tert-pentyl chloride, chloroisopentane, dichloropentane, methylene chloride, 1,5-dichloropentane, chlorobenzene or o-dichlorobenzene.

2. The method according to claim 1, wherein the stated bromide is dropped in the reaction solution at −10° to 80°.

3. The method according to claim 2, wherein after addition of the stated bromide, the reaction occurs for 3-10 hours at −10° to −80°.

4. The method according to claim 1, wherein the reaction also includes after treatment procedures which consists of filtrating to obtain solids and washing them with solvents.

5. The method according to claim 1, wherein the stated 9,9′-bianthryl is obtained as follows: add anthraquinone as raw material and zinc as reducing agent in glacial acetic acid solution, then batch addition of hydrochloric acid at 70-120°, maintain the temperature unchanged and react, then 9,9′-bianthryl is achieved.

6. The method according to claim 5, wherein after addition of the stated hydrochloric acid, reaction occurs 2-15 hours at 70-120°.

7. The method according to claim 6, wherein after batch addition of hydrochloric acid at 80-110°, the reaction occurs for 2-10 hours at 80-110°.

8. The method according to claim 5, wherein the stated reaction takes place under protection of nitrogen.
